(19) United States
(12) Patent Application Publication
(10) Pub. No.: US 2025/0258089 A1
(43) Pub. Date: Aug. 14, 2025

(54) CORRELATED INTERFERENCE POLARIZATION SPECTROMETER

(71) Applicant: Heath Consultants Incorporated, Houston, TX (US)

(72) Inventors: Euan Smith, Cambridgeshire (GB); Vineet Aggarwal, Richmond, TX (US)

(73) Assignee: Heath Consultants Incorporated, Houston, TX (US)

(21) Appl. No.: 19/053,260

(22) Filed: Feb. 13, 2025

Related U.S. Application Data
(60) Provisional application No. 63/552,996, filed on Feb. 13, 2024.
(60) Provisional application No. 63/552,978, filed on Feb. 13, 2024.

(51) Int. Cl. G01N 21/31; G01N 33/00

(52) U.S. Cl. CPC G01N 21/31; G01N 33/0027

(57) ABSTRACT

An apparatus and method of determining the concentration of first and second substances within a sample by exposing the sample to radiation and filtering the radiation transmitted from the sample using a first filter having a number of pass bands at wavelengths corresponding to absorption peaks in a desired quasi-periodic absorption spectrum of the first substance to be detected, and modulating the wavelengths of the pass bands of the first filter. Additionally, filtering the radiation transmitted from the sample using a second filter having a number of pass bands at wavelengths corresponding to absorption peaks in a desired quasi-periodic absorption spectrum of the second substance to be detected, and modulating the wavelengths of the pass bands of the second filter. The concentration of the first and second substances is determined by determining the difference in the maximum and the minimum intensities of the radiation transmitted by the sample.

### CROSS-REFERENCE TO RELATED APPLICATIONS

This application claims the benefit of U.S. Provisional Application Ser. No. 63/552,996 filed on Feb. 13, 2024 and U.S. Provisional Application Ser. No. 63/552,978 filed on Feb. 13, 2024. Applicant incorporates by reference herein Application Ser. Nos. 63/552,996 63/552,978 in their entireties.

### BACKGROUND OF THE INVENTION

### 1. Field of Invention

The present invention relates generally to an optical absorption gas detector and more particularly to an improved correlated interference polarization spectrometer for determining the concentration of a substance within a sample, and in particular for the optical detection of one or more gases within a sample.

### 2. Description of the Related Art

Various optical absorption gas detection techniques based on the measurement of absorption of incident radiation by gas molecules exist. One known type of optical absorption gas detector for determining the concentration of a substance within a sample, for example a gas within a sample, is a correlated interference polarization spectrometer (“CIPS”). A few examples of CIPS devices are disclosed in the following U.S. patents assigned to Bah Holdings, LLC: U.S. Pat. No. 7,030,990 titled “Controlled Interference Spectrometer;” U.S. Pat. No. 7,253,896 titled “Filter;” and U.S. Pat. No. 7,570,360 titled “Optical Absorption Spectrometer and Method for Measuring Concentration of a Substance.” Applicant incorporates by reference U.S. Pat. Nos. 7,030,990, 7,253,896, and 7,570,360 in their entireties.

As is well known and described in the above-referenced Bah Holdings' patents, a CIPS is an optical absorption gas detector that works on the principle that for any given wavelength incident radiation, the quantity that is absorbed is a function of the “cross section” (σ(λ)) (cm²) of any particular molecule in the gas (i.e., the amount of absorption depends on the wavelength). U.S. Pat. No. 7,570,360, col. 1, lines 28-35 states: “If the dependence of the cross-section on the wavelength is very pronounced, then the gas molecules will absorb radiation over a very narrow waveband. Accordingly, the spectral displacement between the maximum and minimum intensities of the transmitted radiation is very small. By measuring the difference between the maximum and minimum intensities of the transmitted radiation, it is possible to calculate the concentration of the gas.”

FIG. 1 is an example of a graph showing the absorption spectrum of methane (CH₄) around the range of 3.20 to 3.30 microns. The spectrum consists of a number of very narrow (approximately 1 nm wide) quasi-periodic absorption bands, labelled 2. The quasi-periodical structure of the electronic absorption spectrum occurs due to the vibrational-rotational properties of the gas molecules.

FIG. 2 is a figure from the aforementioned Bah Holdings patents which describe an exemplary prior art CIPS for detecting the quasi-periodic structure of a gas molecules' absorption spectrum.

As understood by one skilled in the art and with reference to FIG. 2, the conventional CIPS includes a radiation source 11, as for example an incandescent lamp or an LED, positioned next to a collimator 12 for converting the incident radiation into a collinear beam which then passes through a cavity or gas cell 13 containing the gas to be detected. After passing through the cavity 13, the transmitted radiation passes through a bandpass filter 14 which only allows wavelengths in the vicinity of the desired absorption spectra to pass.

Following the bandpass filter 14 is a controlled interference polarization filter (cIPF) 15 which generates a transmission spectrum consisting of very narrow pass bands coinciding with the quasi-periodic absorption spectrum of the target gas. As known in the prior art, the cIPF 15 is controlled to cause the transmission spectrum to be scanned back and forth within the selected working range. The operation of the cIPF 15 is controlled by a power supply 17 which drives the cIPF 15 and a detection system. The detection system includes a detector 16, an amplifier 18, a microprocessor 19 and a display 20.

The conventional CIPS detects the quasi-periodic structure by filtering radiation transmitted by the sample gas in the cavity 13 using a comb filter which is generated by the cIPF 15. As disclosed in the Bah Holdings patents:

“The cIPF is formed from a modified interference polarisation filter (IPF) which uses the phenomenon of birefringence in certain crystals to obtain a transmission spectrum which is characterised by a quasi-periodic sequence of spectral passbands. In order to be able to use the IPF in the detection of gases, the IPF must provide a transmission spectrum that closely matches the quasi-periodic absorption spectra of the gas to be detected (i.e. the bandwidth between adjacent peaks in the absorption spectrum of the gas to be detected must correspond to the bandwidth between adjacent transmission peaks in the IPF transmission spectrum). Furthermore, the IPF must be able to shift this spectrum in time so that one can detect the intensity of the radiation transmitted at both the absorption and non-absorption bands of the absorption spectrum of the target gas.” U.S. Pat. No. 7,750,360, col. 1, lines 47-62.

As disclosed in U.S. Pat. No. 7,030,990 at col. 1, lines 54-58, the IPF may be modulated by (1) changing the width of the natural birefringent section; (2) changing the type of birefringent material used; or (3) introducing an additional birefringent section. The '990 patent further discloses various ways that spectral scanning of the radiation passing through an IPF can be accomplished, among them being: a) by mechanically rotating the output polarizer by 90° with respect to the optical axis of the birefringent crystal; b) using nematic liquid crystals together with an IPF to achieve tuneability; or c) using a Photoelastic Modulator of Polarization (PMP), explained in further detail in the '990 patent.

It is desired to have a CIPS capable of detecting more than one target gas in a sample. It is further desirable to have a CIPS capable of detecting at least two target gases in a sample, more specifically, capable of detecting methane and ethane in a sample. It is also desirable to have a CIPS capable of detecting at least two target gases in a sample at very low concentrations.

### SUMMARY OF INVENTION

One aspect of the invention is an apparatus and method of determining the concentration of first and second substances within a sample by exposing the sample to radiation and filtering the radiation transmitted from the sample using a first filter having a number of pass bands at wavelengths corresponding to absorption peaks in a desired quasi-periodic absorption spectrum of the first substance to be detected, and modulating the wavelengths of the pass bands of the first filter. Additionally, filtering the radiation transmitted from the sample using a second filter having a number of pass bands at wavelengths corresponding to absorption peaks in a desired quasi-periodic absorption spectrum of the second substance to be detected, and modulating the wavelengths of the pass bands of the second filter. The concentration of the first and second substances is determined by determining the difference in the maximum and the minimum intensities of the radiation transmitted by the sample. One aspect of an embodiment of the invention is the use of dual crystals on a single optical path for sensitive methane and ethane detection for natural gas discrimination exploiting the similar quasi-periodic property of methane and ethane absorption lines in nearby, but slightly different, mid-IR bands to minimize cross-talk.

In one aspect of the invention, a correlated interference polarization spectrometer for determining the concentration of first and second substances within a sample comprises a radiation source for supplying radiation to a sample to be measured and a gas cell adapted to contain the sample. A controlled interference polarization filter section filters radiation transmitted by the sample and the controlled interference polarization filter section comprises first and second filters. The first filter includes a first bandpass filter allowing only wavelengths in the vicinity of the desired absorption spectra of the first substance to pass and a first birefringent crystal having transmission peaks corresponding to desired quasi-periodic absorption peaks of the absorption spectra of the first substance. The second filter comprises a second bandpass filter allowing only wavelengths in the vicinity of the desired absorption spectra of the second substance to pass and a second birefringent crystal having transmission peaks corresponding to desired quasi-periodic absorption peaks of the absorption spectra of the second substance. A detector assembly comprising a radiation detector detects the filtered radiation and generates a radiation signal in accordance with the detected radiation.

One aspect of an embodiment of the invention is the use of dual crystals on a single optical path for sensitive methane and ethane detection for natural gas discrimination exploiting the similar quasi-periodic property of methane and ethane absorption lines in a nearby but slightly different mid-IR bands to minimize cross-talk.

One aspect of an embodiment of the invention is the use of angular tuning of the methane birefringent crystal and thermal tuning of the ethane birefringent crystal to provide sensitivity to both on the same gas sample to enhance natural gas discrimination.

### BRIEF DESCRIPTION OF THE DRAWINGS

Some of the features and benefits of the present invention having been stated, others will become apparent as the description proceeds when taken in conjunction with the accompanying drawings, in which:

FIG. 1 is a graph showing the absorption spectrum of methane in the range of approximately 3.20 to 3.30 microns;

FIG. 2 is a schematic diagram of an example of a prior art CIPS device;

FIG. 3 is a schematic diagram of an example of one type of interference polarization filter (IPF);

FIG. 4 is an example of graphs of the cIPF transmission spectrum positioned below the methane absorption spectrum;

FIG. 5 are graphs of the absorption spectrum for methane and ethane;

FIG. 6 are graphs showing the operation of methane and ethane birefringent crystals and the CIPS fringes from the ethane and methane birefringent crystals;

FIG. 7 is a schematic diagram of a first embodiment of an enhanced CIPS device;

FIG. 8 is a schematic diagram of a second embodiment of the enhanced CIPS device;

FIG. 9 is a schematic diagram of a third embodiment of the enhanced CIPS device;

FIG. 10A is simplified schematic of a birefringent crystal, light beam, lens and image plane showing the birefringent crystal normal to the incoming beam;

FIG. 10B is similar to FIG. 10A but showing the birefringent crystal angularly tuned by rotating the crystal perpendicular to the light beam propagation direction; and

FIGS. 10C to 10E are similar to FIGS. 10A and 10B, but showing the birefringent crystal split in two parts.

### DETAILED DESCRIPTION OF INVENTION

The method and system of the present disclosure will now be described more fully hereinafter with reference to the accompanying drawings in which embodiments are shown. The method and system of the present disclosure may be in many different forms and should not be construed as limited to the illustrated embodiments set forth herein; rather, these embodiments are provided so that this disclosure will be thorough and complete, and will fully convey its scope to those skilled in the art. Like numbers refer to like elements throughout. In an embodiment, usage of the term “about” includes +/−5% of a cited magnitude. In an embodiment, the term “substantially” includes +/−5% of a cited magnitude, comparison, or description. In an embodiment, usage of the term “generally” includes +/−10% of a cited magnitude.

It is to be further understood that the scope of the present disclosure is not limited to the exact details of construction, operation, exact materials, or embodiments shown and described, as modifications and equivalents will be apparent to one skilled in the art. In the drawings and specification, there have been disclosed illustrative embodiments and, although specific terms are employed, they are used in a generic and descriptive sense only and not for the purpose of limitation.

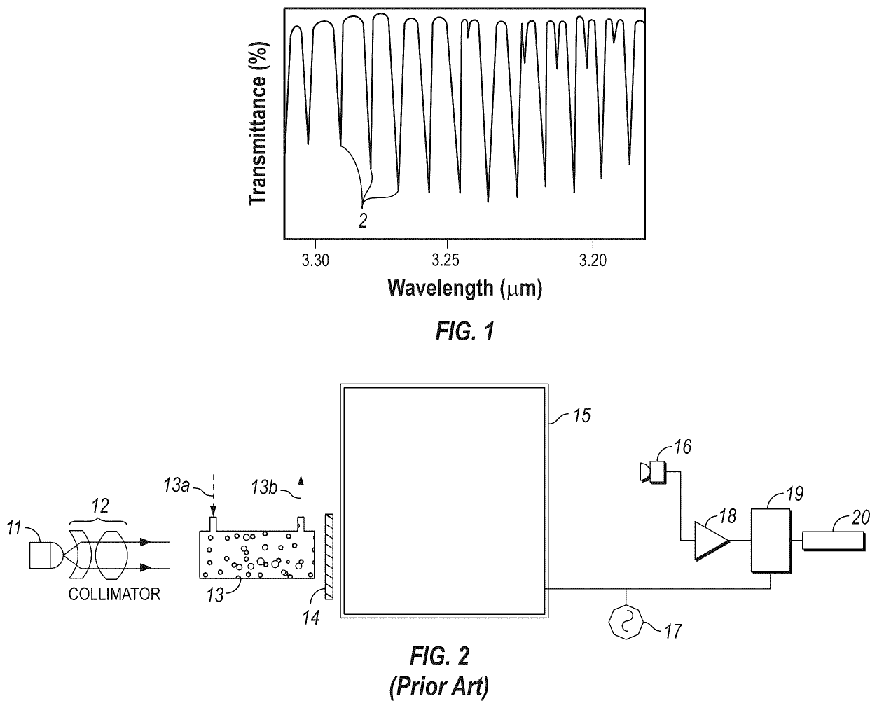

As discussed above in the Background of the Invention, a CIPS for detecting a single target gas, as for example methane, is well known in the art. FIG. 2 shows an example of a correlated interference polarization spectrometer (CIPS) capable of detecting the quasi periodic structure of the target gas molecules' absorption spectrum. FIG. 1 shows the absorption spectrum of methane in the range of approximately 3.20 to 3.30 microns.

Referring to FIG. 2, the collimator 12 converts the incident radiation from the radiation source 11 into a collinear beam which then passes through the cavity or gas cell 13 containing the target gas to be detected, the cavity 13 having inlet and outlet openings 13a and 13b. After the incident radiation passes through the cavity 13, the transmitted radiation passes through a bandpass filter 14 which only allows wavelengths in the vicinity of the desired absorption spectra to pass.

The CIPS includes a controlled interference polarization filter (cIPF) section 15 which is adapted to generate a transmission spectrum that comprises very narrow pass bands which coincide with the quasi periodic absorption spectrum of the target gas. The power supply 17 and processor 19 control the cIPF 15 to scan the transmission spectrum back and forth within the selected working range. The detector 16, coupled to the amplifier 18, detects signals which are then amplified by the amplifier 18. The output from the amplifier 18 is received by the processor 19 which is coupled to the power supply 17. The processing of the detected signals is synchronized with the operation of the cIPF 15 and the processed signals are directed to the display 20 which displays an indication of the target gas concentration.

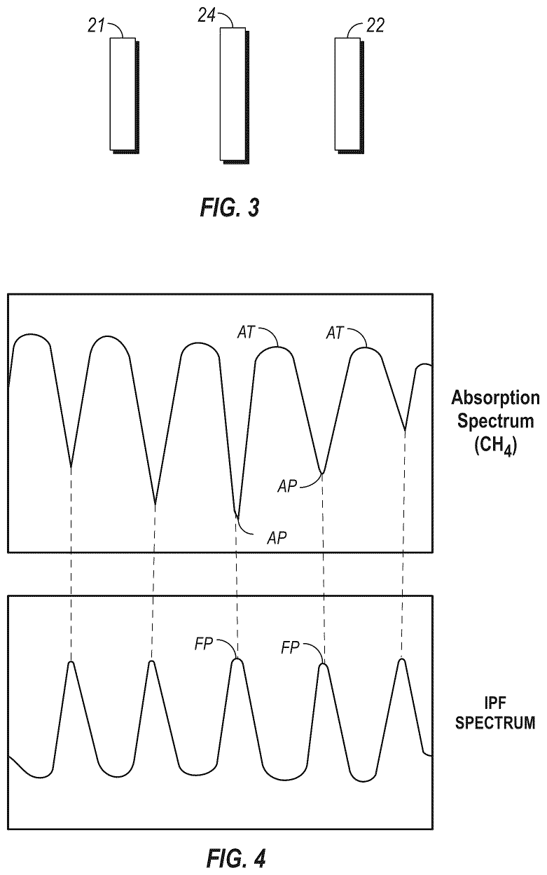

The cIPF 15 is a modified interference polarization filter which uses birefringence in particular crystals to obtain a transmission spectrum characterized by a quasi-periodic sequence of spectral pass bands. As shown in FIG. 3, an interference polarization filter may include input and output polarizers 21 and 22 and a birefringent crystal 24. The interference polarization filter preferably includes a modulator to cause the transmission spectrum to be scanned back and forth within the selected working range.

FIG. 4 is a depiction of an example of the cIPF transmission spectrum positioned below the methane absorption spectrum. The cIPF transmission spectrum includes a number of transmission peaks FP. The cIPF acts as a comb filter. By using an appropriate crystal, the transmission peaks FP can be selected to correspond to the absorption peaks AP of the absorption spectrum of the target gas, in this example methane (CH₄). As is well understood in the art, when the peaks FP and AP are aligned as shown in FIG. 4, the cIPF transmitted radiation will be the radiation having wavelengths which are absorbed by the target gas, methane. The comb filter scans back and forth between right and left and when the transmission peaks FP are aligned with troughs AT of the absorption spectrum, then the cIPF transmitted radiation represents wavelengths which are transmitted by the target gas. As a result, maximum and minimum transmission intensities in the absorption spectrum of the gas can be determined and used to generate a concentration measurement of the target gas.

In the present invention, the previously described CIPS has been modified to provide various enhancements and features. In one or more embodiments, the enhanced CIPS is able to detect more than a single target gas and is capable of detecting at least two target gases. In one or more embodiments the enhanced CIPS is adapted to detect ethane and methane. Another aspect of the enhanced CIPS is that it can detect the target gases at very low concentrations.

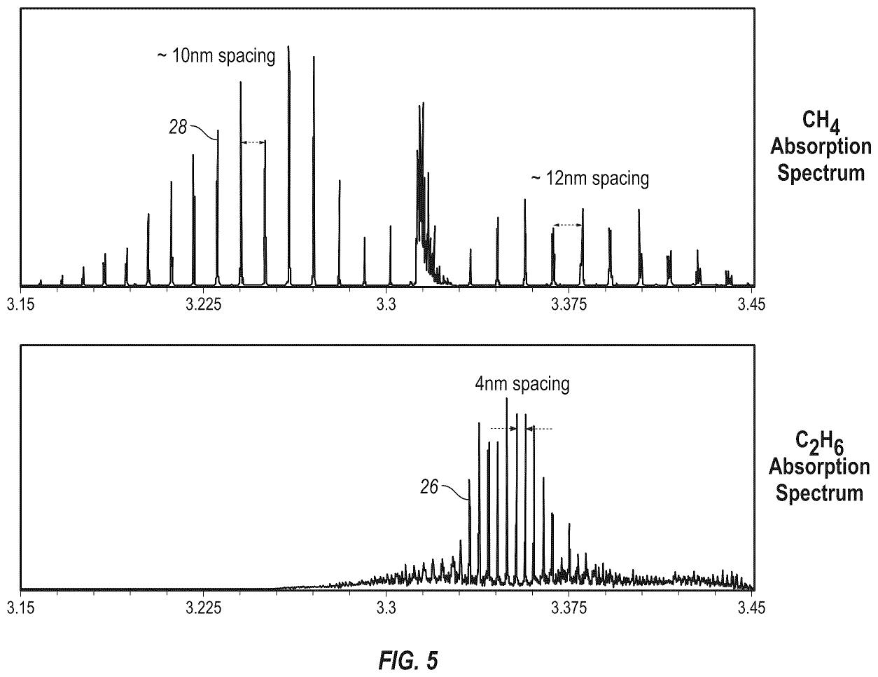

The absorption spectrums for methane and ethane 28 and 26, respectively, are shown in FIG. 5, with the upper graph being the absorption spectrum for methane (CH₄) and the lower graph for ethane (C₂H₆). As shown in FIG. 5, the methane absorption spectrum 28 overlaps the ethane absorption spectrum 26. Methane and ethane each have a quasi periodic sequence of spectral pass bands (i.e., spacing between peaks is nearly uniform). For methane, the peaks are ˜10 nm apart in the 3150-3300 nm range and ˜12 nm apart in the 3335-3440 nm range. The spacing of the peaks for ethane is much smaller—˜4 nm—in the range of 3330-3375 nm.

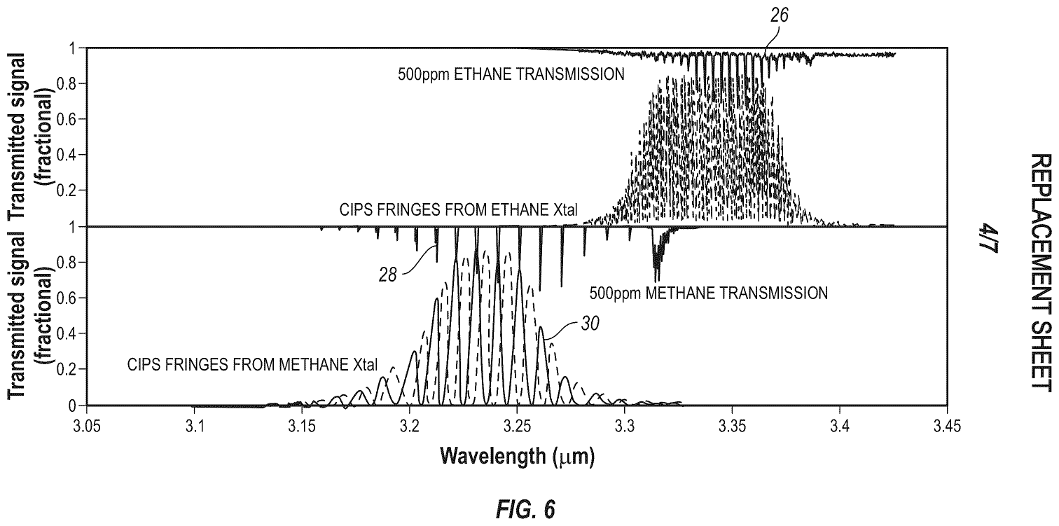

As discussed above with respect to FIG. 5, the methane absorption spectrum 28 overlaps the ethane absorption spectrum 26. FIG. 6 are graphs illustrating the operation of methane and ethane crystals of the enhanced CIPS discussed below. With reference to FIG. 6, the upper graph shows the ethane absorption spectrum 26 having downwardly extending peaks spaced ˜4 nm apart and the methane absorption spectrum 28 having downwardly extending peaks spaced ˜10 nm apart in the lower graph. The difference in the spacing between the peaks for methane and ethane is readily apparent. The plots having upwardly extending peaks in the graphs represent the enhanced CIPS transmission spectra 30. In each plot a dashed line is shifted to the left and right of the solid line to represent shifting of the CIPS transmission spectra 30 as discussed above.

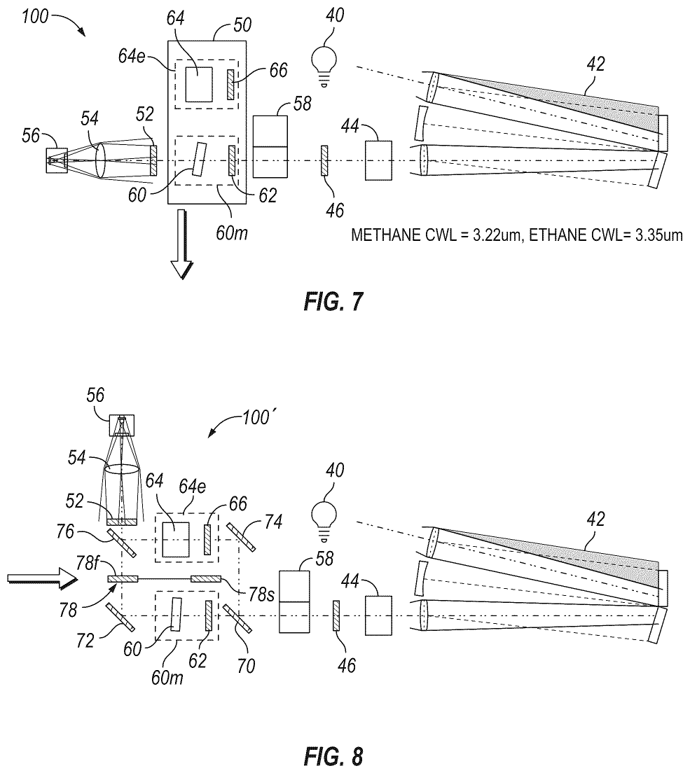

With reference to FIGS. 7-9, the enhanced CIPS includes one birefringent crystal 64 selected to generate a transmission spectrum having peaks separated by ˜4 nm—the peak spacing for ethane, while a second birefringent crystal 60 is selected to generate a transmission spectrum having peaks separated by ˜10 nm.

FIGS. 7-9 illustrate a few embodiments of the enhanced CIPS for detecting a plurality of gases, and more particularly for detecting methane and ethane in a compact, small handheld apparatus. It is to be understood that the invention is not limited to the detection of methane and ethane; however, the following description will discuss the invention with reference to methane and ethane. Although not shown in FIGS. 7-9, it is to be understood that the enhanced CIPS preferably includes a power supply 17, an amplifier 18, a microprocessor 19 and a display 20 similar to that shown and described above with respect to FIG. 2.

FIG. 7 is a schematic diagram of a first embodiment of an enhanced CIPS device generally referenced as 100. The enhanced CIPS 100 comprises a radiation source 40, preferably an incandescent lamp, transmitting light to a gas cell 42, preferably a White cell, containing an ambient gas sample to be tested for the detection of the target gases. It is to be understood that a White cell 42 is commonly used in optical systems and includes spherical mirrors which act like lenses and repeatedly image the beam. The White cell 42 is especially suited for providing a long optical path in a small sample volume. Usage of the White cell 42 aids in reducing the size of the enhanced CIPS 100. It is to be understood that while use of a White cell is desirable, the invention is not so limited.

The transmitted light exits the White cell 42 and passes through an optional calibration cell or Cal cell 44 when calibrating the device 100. During calibration of the enhanced CIPS device 100, the Cal cell 44, positioned in the path of the radiation source light, includes a calibration vial of gas of a known concentration. The gas vial and optionally the Cal cell 44 is removed from the light path during operational use of the enhanced CIPS 100 to detect the target gases in an ambient sample. In one embodiment, the Cal cell 44 contains a known concentration of methane for calibrating the methane detection system and alternately a known concentration of ethane for calibrating the ethane detection system. As shown in FIG. 7, after passing through the White cell 42 (and the optional Cal cell 44 during calibration), the light passes to a controlled interference polarization filter section 50 comprising input and output polarizers 46 and 52, respectively, an acousto-optic (AO) modulator 58, a first combination 60m of birefringent crystal 60 and bandpass filter 62 for methane and a second combination 64e of birefringent crystal 64 and bandpass filter 66 for ethane. With reference to FIG. 7, the light passes through the input polarizer 46 and the AO modulator 58, one of the first and second combinations 60m, 64e, the output polarizer 52, and a lens 54 before being received at the detector 56 of the detection system.

In the illustrated first embodiment of the enhanced CIPS 100, the light exiting the White cell 42 preferably has a straight path directed to the detector 56. As discussed above and known in the art, the AO modulator 58 provides for the shift of the transmission spectrum relative to the absorption spectrum of the target gas. The first and second combinations 60m and 64e, respectively, are controlled and switched to be positioned into the path of the light at discreet separate time periods. The combination switching may be done mechanically, electro-mechanically and other ways as known to one skilled in the art. Merely by way of example, the switching may be done using a lever mechanism, a solenoid and various other means. The bandpass filters 62 and 66 only allow wavelengths in the vicinity of the desired absorption spectra to pass. Thus, the bandpass filter 62 only allows wavelengths in the vicinity of the desired methane absorption spectra to pass and the bandpass filter 66 only allows wavelengths in the vicinity of the desired ethane absorption spectra to pass. For example, the bandpass filter 62 allows only wavelengths in the 3.22 micron region to pass for methane, whereas the bandpass filter 66 allows only wavelengths in the 3.35 micron region to pass for ethane. The methane center wavelength is 3.22 microns and the ethane center wavelength is 3.35 microns. The bandpass filters 62 and 66 may have a 100 nm bandwidth.

FIG. 7 shows the first combination 60m of the bandpass filter 62 and the birefringent crystal 60 of the cIPF section 50 in a first position in the path of the light beam for the detection of methane. In this first position, none of the light beam is passing through the bandpass filter 66 and birefringent crystal 64. In a second position (not shown), the second combination 64e of the bandpass filter 66 and birefringent crystal 64 are moved in the path of the light beam for the detection of ethane. It is to be understood that in the second position the bandpass filter 62 and the birefringent crystal 60 are not in the path of the light beam directed to the detector 56. At controlled and predetermined times the position of the combinations 60m, 64e of bandpass filter and birefringent crystal are switched to separately detect the two target gases. The microprocessor, similar to processor 19 (FIG. 2) may control the serial timing and switching of the first and second combinations 60m and 64e, respectively.

A second embodiment of the enhanced CIPS, generally referenced as 100′, for detecting two gases, preferably ethane and methane, is shown in FIG. 8 and includes the same or similar radiation source 40, gas or White cell 42, Cal cell 44, input polarizer 46, output polarizer 52, lens 54, detector 56 and AO modulator 58. Additionally, the same first and second combinations 60m, 64e of birefringent crystal 60, 64 and bandpass filter 62, 66, respectively, are utilized.

In the second embodiment of the enhanced CIPS 100′, a first dichroic mirror 70 is positioned in the path of the light from the White cell 42. A portion of the light is reflected by the first dichroic mirror 70 and a portion of the light is simultaneously transmitted through the first dichroic mirror 70. In the illustrated case, the lower bandwidth wavelength is reflected and the higher bandwidth wavelength is transmitted by the first dichroic mirror 70. The transmitted bandwidth wavelength passes through the methane bandpass filter 62 and birefringent crystal 60 and is reflected from a second dichroic mirror 72 in the direction of a fourth dichroic mirror 76, through the fourth dichroic mirror 76 to the output polarizer 52, the lens 54 and to the detector 56.

The light reflected from first dichroic mirror 70 is directed to and reflected off a third dichroic mirror 74 to the ethane bandpass filter 66 and birefringent crystal 64 and then reflected off the fourth dichroic mirror 76, through the output polarizer 52, the lens 54 and to the detector 56.

In the second embodiment of the enhanced CIPS 100′, a shutter or pair of shutters 78 may be used to block the light transmitted through the first dichroic mirror 70 or alternatively to block the light reflected from the first dichroic mirror 70. In FIG. 8, a first shutter 78f is shown positioned in the path of the light transmitted through the first dichroic mirror and reflected from the second dichroic mirror 72 while a second shutter 78s is not in and removed from the path of the light reflected from the first dichroic mirror 70. In this shutter position, the enhanced CIPS 100′ is able to conduct the ethane analysis. Although not shown, it is to be understood that the first and second shutters 78f and 78s, respectively, may be repositioned such that the second shutter 78s is positioned in the path of the light reflected from the first dichroic mirror 70 while the first shutter 78f is not in and removed from the path of the light transmitted through the first dichroic mirror 70. In this shutter position, the enhanced CIPS 100′ is able to conduct the methane analysis. It is to be understood that alternatively a single shutter 78 could be used by switching it from the path of the light transmitted through the first dichroic mirror 70 to the path of the light reflected from the first dichroic mirror.

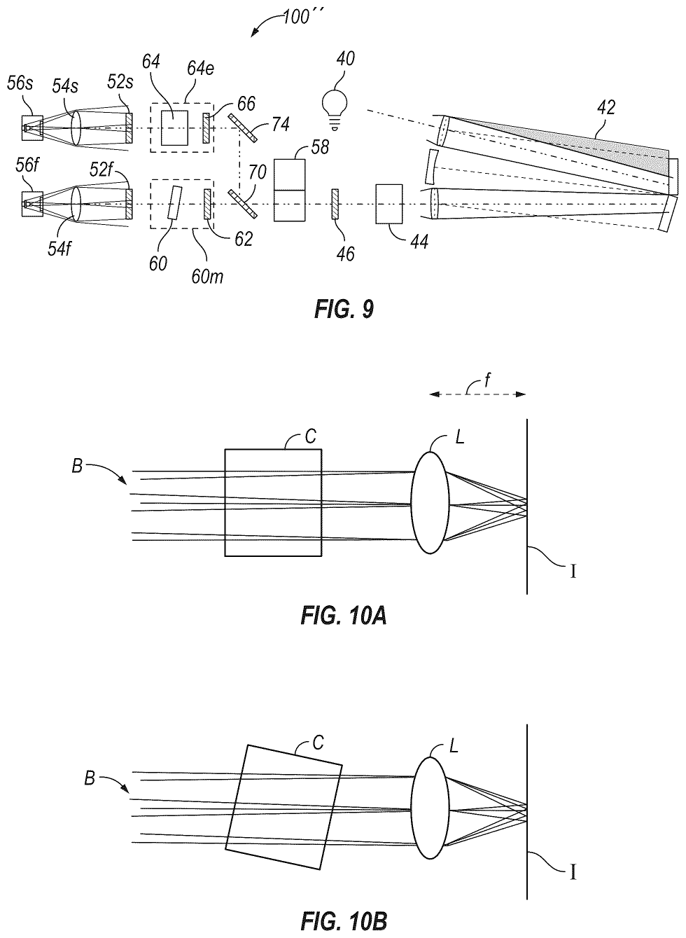

A third embodiment of the enhanced CIPS for detecting ethane and methane, generally referenced as 100″, is shown in FIG. 9. This embodiment requires two detectors 56f, 56s, two lenses 54f, 54s, and two output polarizers 52f, 52s. Light from radiation source 40 passes through the White cell 42, the input polarizer 46, and the AO modulator 58 and is partially transmitted through and partially reflected by a first dichroic mirror 70. The transmitted bandwidth wavelength passes through the first combination 60m of the methane bandpass filter 62 and birefringent crystal 60 and then on through the first output polarizer 52f, the first lens 54f and to the first detector 56f.

The light reflected from the first dichroic mirror 70 is directed to and reflected off a second dichroic mirror 74 to the second combination 64e of the ethane bandpass filter 66 and birefringent crystal 64 and then on through the second output polarizer 52s, the second lens 54s and to the second detector 56s. It is to be understood that alternatively the arrangement and positioning of the combination 64e of bandpass filter 66 and birefringent crystal 64, the second output polarizer 52s, second lens 54s and second detector 56s be in the path of the light reflected from the first dichroic mirror 70. In such an arrangement the second dichroic mirror 74 could be omitted.

In the first and second embodiments of the enhanced CIPS 100 and 100′, a single detector 56 is utilized and switching occurs in order to perform the ethane analysis and methane analysis. The first embodiment 100 switches the position of the combinations of bandpass filter and birefringent crystal into the single path of the light beam from the gas cell 42, whereas the second embodiment 100′ switches the position of the shutter(s) 78 and uses dichroic mirrors 70, 72, 74 and 76 to split the light beam from the gas cell 42 according to certain bandwidth wavelengths. In the third embodiment 100″, first and second detectors 56f, 56f are utilized in conjunction with one or more dichroic mirrors 70, 74.

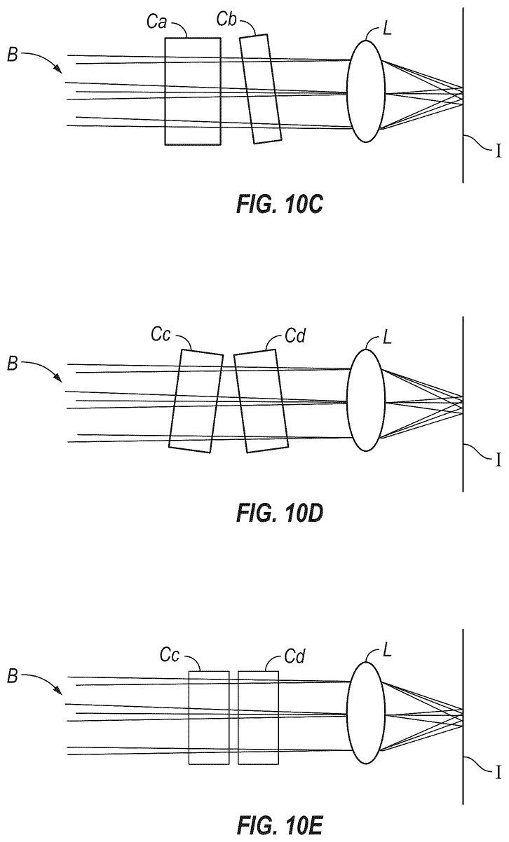

Another aspect that may be incorporated in any of the embodiments of the enhanced CIPS 100, 100′, 100″ is with respect to the tuning system. A conventional CIPS rotates the crystal or birefringent crystal to tune the system for methane detection. Simplified illustrations of this are shown in FIGS. 10A and 10B. In FIG. 10A, a birefringent crystal C is positioned normal to an incoming light beam B. After passing through the crystal C the light beam passes through the lens L and is directed to an image plane I at the detector. FIG. 10B shows the birefringent crystal C angularly tuned by rotating the crystal C perpendicular to the light beam propagation direction.

The enhanced CIPS 100, 100′, 100″ uses or may use the conventional technique for methane; however, preferably uses a different technique with respect to ethane. As discussed above, the crystal 64 for ethane is different than the crystal 60 for methane. This is primarily due to the close spacing between the ethane peaks. The crystal 60 for methane has a thickness of ˜7 mm whereas for ethane the crystal 64 has a thickness of ˜21 mm. Preferably, the tuning of the crystal 64 for ethane is done thermally by adjusting the temperature of the crystal 64. The reasons for this are discussed below.

Beam divergence complications with Ethane CIPS fringes.

While the basic operation of a CIPS system for methane detection and ethane detection is substantially the same for each, there are implications of the closer fringe spacing required for ethane which have an impact on device operation.

To tune a CIPS system to get a maximum detection signal, the thickness of the birefringent crystal 60 the optical beam passes through needs to be tuned. This tuning is typically achieved by rotating the crystal 60 perpendicular to the beam propagation direction, thereby increasing the thickness of the crystal 60 the light passes though by 1/cos θ where θ is the adjusted angle, as for example illustrated in FIG. 10B. As the crystal 60 is rotated the signal will pass through regions of maximum and minimum signal. Generally for purposes of this discussion, the signal detected by the detector 56, 56f will be the integral of the maximum and minimum signals over the beam divergence signal. As the crystal 60 is rotated the divergence circle will start to average over both areas of maximum and minimum signal until, at some larger angle, the maximum and minimum signals will cancel out resulting in no net detected signal. For the methane system that angle is large, so while there is an advantage in keeping the tuning angle small, it does not present a substantial impediment to the operation of the methane system. However, for the ethane crystal 64 the fringes are 3-times closer together in wavelength space and angular space. The risk for the ethane system is that the angular spread of the beam will substantially degrade the CIPS signal for anything other than very small angular adjustments, making angular tuning of the ethane detection crystal 64 highly disadvantageous.

There are a few solutions of this (and these solutions may also be used for the methane crystal):
1) The effective degree of birefringence through the crystal changes with temperature, both due to thermal expansion and temperature dependent refractive index. Keeping the ethane crystal 64 fixed (as opposed to rotatable) and normal to the incoming beam as for example illustrated in FIG. 10A, the crystal 64 can be tuned via adjustment of the temperature at which the crystal 64 is held. It is well known in the art to incorporate a thermometer and heater/cooler to control the temperature of the birefringent crystal. A heater has also been used in the prior art to remove condensation. In cases where both the ethane and methane detection crystals 64 and 60, respectively, are in a single oven (e.g., a single confined space) the ethane crystal 64 could be initially temperature tuned and then the methane crystal 60 tuned using angle (i.e. rotating the crystal 60).
2) The ethane detection crystal 64 can be split into two parts, preferably one thinner and one thicker, and the thinner crystal section can be used for angle tuning. FIG. 10C is an illustration of a two part birefringent crystal comprised of parts Ca and Cb in which the thicker part Ca is fixed and normal to the light beam B and the thinner part Cb is tuned by rotating. This reduces, although does not remove, the fringe averaging of rotating the whole crystal.
3) The ethane detection crystal 64 can be split into two equal thicknesses and the two crystals rotated in opposite directions to each other. FIG. 10D is an illustration of a two part birefringent crystal comprised of parts Cc and Cd. The crystal parts Cc and Cd may have equal thicknesses and each is rotated in opposite directions to each other for tuning. The shift in the bullseye pattern caused by the rotation of one crystal Cc is substantially reversed by the rotation of the second crystal Cd.
4) Splitting the ethane detection crystal 64 similarly to 2 or 3 above, but rotating one or both crystals about the axis of the beam propagation in order to tune the CIPS effect. FIG. 10E is an illustration of a two part crystal comprised of parts Cc and Cd in which one of both of the crystal parts Cc, Cd may be rotated about the axis of the light beam B propagation direction (i.e., in directions into and out of the plane of the paper).
5) Use of an external lower birefringent crystal material to tune the system, using any of the methods from 2-4 above.
6) As 5 above and where the material chosen is less sensitive to temperature effects and therefore has less need for thermal control.
7) When the ethane detection crystal 64 is in use, or for the ethane detection crystal beam path, take measures to reduce the beam divergence of the light passing through the crystal 64 which will be incident on the detector 56, 56s—this is less desired as it will also reduce the overall signal, but may be preferred for other reasons. This could be achieved by, for example, using a detector 56, 56s with a smaller detection area, or by introducing an aperture in the plane of the detector 56, 56s. Other methods will be apparent to a skilled optical engineer.

The close fringes in angular space also cause a second issue for ethane detection, which is that small mechanical changes in the system (for example due to thermal changes in the mechanics as the system warms up) can cause a greater change in the observed CIPS signal for ethane than is the case for methane detection. This is typically described as system drift, and this can have a very large impact on the ethane detection performance.

One way to compensate for this is through process, ensuring that there will have been little time between an assessment of the current ethane detection performance and the assay of a sample. The sequence of steps desired to get the best out of the system are as follows.
1) Before any assay of ethane content of a sample, for example a sample of the ambient environment, the system response of both the ethane and methane detection systems is calibrated using a calibration vial of gas of a known concentration which can be inserted into the beam path, as for example the Cal cell 44. This procedure of field calibration is known and standard for CIPS-based gas detection devices.
2) After calibration a sample is drawn into the gas cell 42 of the instrument 100, 100′, 100″ and then the pump turned off.
3) Both ethane and methane CIPS detection channels, either serially (if switched) or in parallel (if simultaneously operable) will be used to assess the contained sample. In particular this measurement step must take place a short amount of time, such as less than 5 minutes, after field calibration to ensure the system has not had time to drift to any significant degree.

### NOMENCLATURE

absorption peaks AP
troughs AT
light beam B
birefringent crystal C, Ca, Cb, Cc, Cd
transmission peaks FP
image plane I
lens L
absorption bands (methane) 2
radiation source 11
collimator 12
cavity 13
inlet opening 13a
outlet opening 13b
bandpass filter 14
controlled interference polarization filter (cIPF) 15
detector 16
power supply 17
amplifier 18
microprocessor 19
display 20
input polarizer 21
output polarizer 22
birefringent crystal 24
ethane absorption spectrum 26
methane absorption spectrum 28
enhanced CIPS transmission spectra 30
radiation source 40
gas cell 42
calibration cell 44
input polarizer 46
controlled interference polarization filter (cIPF) section 50
output polarizer 52
first output polarizer 52f
second output polarizer 52s
lens 54
first lens 54f
second lens 54s
detector 56
first detector 56f
second detector 56s
acousto-optic (AO) modulator 58
birefringent crystal (methane) 60
first combination 60m (birefringent crystal/bandpass filter (methane))
bandpass filter (methane) 62
birefringent crystal (ethane) 64
second combination 64e (birefringent crystal/bandpass filter (methane))
bandpass filter (ethane) 66
first dichroic mirror 70
second dichroic mirror 72
third dichroic mirror 74
fourth dichroic mirror 76
pair of shutters 78
first shutter 78f
second shutter 78s
enhanced CIPS 100, 100′, 100″

The present invention described herein is well adapted to carry out the objects and attain the ends and advantages mentioned, as well as others inherent therein. While a presently preferred embodiment of the invention has been given for purposes of disclosure, numerous changes exist in the details of procedures for accomplishing the desired results. Although the invention has been described with reference to exemplary embodiments, it should be appreciated by those of skill in the art that various modifications are well within the scope and spirit of this disclosure. Further, those of skill in the art will appreciate that the invention is not limited to any specific embodiment and/or application and that the various embodiments described herein are illustrative and not restrictive.

What is claimed is:

1. A method of determining the concentration of first and second substances within a sample, the method comprising the steps of:
exposing the sample to radiation;
filtering the radiation transmitted from the sample using a first filter having a number of pass bands at wavelengths corresponding to absorption peaks in a desired quasi-periodic absorption spectrum of the first substance to be detected, and modulating the wavelengths of the pass bands of the first filter; and
filtering the radiation transmitted from the sample using a second filter having a number of pass bands at wavelengths corresponding to absorption peaks in a desired quasi-periodic absorption spectrum of the second substance to be detected, and modulating the wavelengths of the pass bands of the second filter;
detecting the filtered radiation of the first filter, the detection being performed in accordance with the modulation of the first filter to determine the difference in the maximum and the minimum intensities of the radiation transmitted by the sample to thereby determine the concentration of the first substance; and
detecting the filtered radiation of the second filter, the detection being performed in accordance with the modulation of the second filter to determine the difference in the maximum and the minimum intensities of the radiation transmitted by the sample to thereby determine the concentration of the second substance.

2. The method of claim 1, wherein:
the step of filtering the radiation transmitted from the sample using a first filter comprises:
a first bandpass filter allowing only wavelengths in the vicinity of the desired absorption spectra of the first substance to pass; and
a first birefringent crystal having transmission peaks corresponding to the desired quasi-periodic absorption peaks of the absorption spectra of the first substance; and
the step of filtering the radiation transmitted from the sample using a second filter comprises:
a second bandpass filter allowing only wavelengths in the vicinity of the desired absorption spectra of the second substance to pass; and
a second birefringent crystal having transmission peaks corresponding to the desired quasi-periodic absorption peaks of the absorption spectra of the second substance.

3. The method of claim 2, wherein the first substance is methane and the second substance is ethane.

4. The method of claim 3, wherein the first bandpass filter has a center wavelength of 3.22 nanometers and the second bandpass filter has a center wavelength of 3.35 nanometers.

5. The method of claim 3, further comprising the step of rotationally tuning the first birefringent crystal and thermally tuning the second birefringent crystal.

6. The method of claim 2, further comprising the step of:
in the path of the radiation transmitted from the sample, serially switching the position of the combination of the first bandpass filter and first birefringent crystal with the combination of the second bandpass filter and second birefringent crystal.

7. The method of claim 2, further comprising the steps of:
in the path of the radiation transmitted from the sample, directing a first portion of the radiation to the first bandpass filter and first birefringent crystal and directing a second portion of the radiation to the second bandpass filter and second birefringent crystal;
switching at least one shutter to block the first or second portion radiations to allow serially detecting of the filtered radiation of the first filter and second filters.

8. The method of claim 2, further comprising the steps of:
in the path of the radiation transmitted from the sample, directing a first portion of the radiation to the first bandpass filter and first birefringent crystal and directing a second portion of the radiation to the second bandpass filter and second birefringent crystal;
wherein the steps of detecting the filtered radiation of the first filter and detecting the filtered radiation of the second filter occurs concurrently.

9. The method of claim 3, further comprising the step of tuning at least one of the first and second birefringent crystals by dividing the birefringent crystal into two parts, with one part fixed and the other part angularly tuned.

10. The method of claim 9, wherein the birefringent crystal for methane has a first crystal part thicker than a second crystal part, with the first crystal part fixed and the second crystal part angularly tuned.

11. The method of claim 3, further comprising the step of tuning at least one of the first and second birefringent crystals by dividing the birefringent crystal into two parts, and tuning the birefringent crystal by rotating the crystal parts in opposite directions to each other.

12. A correlated interference polarization spectrometer for determining the concentration of first and second substances within a sample, the spectrometer comprising:
a radiation source for supplying radiation to a sample to be measured;
a gas cell adapted to contain the sample;
a controlled interference polarization filter section for filtering radiation transmitted by the sample, the controlled interference polarization filter section comprising first and second filters, the first filter comprising:
a first bandpass filter allowing only wavelengths in the vicinity of the desired absorption spectra of the first substance to pass; and
a first birefringent crystal having transmission peaks corresponding to desired quasi-periodic absorption peaks of the absorption spectra of the first substance;
the second filter comprising:
a second bandpass filter allowing only wavelengths in the vicinity of the desired absorption spectra of the second substance to pass; and
a second birefringent crystal having transmission peaks corresponding to desired quasi-periodic absorption peaks of the absorption spectra of the second substance; and
a detector assembly comprising a radiation detector for detecting the filtered radiation and generating a radiation signal in accordance with the detected radiation.

13. The correlated interference polarization spectrometer of claim 12, wherein the controlled interference polarization filter section further comprises an acoustic-optic modulator adapted to modulate the wavelengths of the pass bands of the first and second filters.

14. The correlated interference polarization spectrometer of claim 12, wherein the controlled interference polarization filter section further comprises input and output polarizers.

15. The correlated interference polarization spectrometer of claim 12, further comprising means for serially switching the position of the combination of the first bandpass filter and first birefringent crystal with the combination of the second bandpass filter and second birefringent crystal in a path of the radiation transmitted from the sample.

16. The correlated interference polarization spectrometer of claim 12, further comprising:
means for directing a first portion of the transmitted radiation to the first bandpass filter and first birefringent crystal and directing a second portion of the radiation to the second bandpass filter and second birefringent crystal; and
switching means to block the first or second portion radiations to allow serially detecting of the filtered radiation of the first filter and second filters.

17. The correlated interference polarization spectrometer of claim 12, further comprising:
means for directing a first portion of the radiation to the first bandpass filter and first birefringent crystal and directing a second portion of the radiation to the second bandpass filter and second birefringent crystal; and
the detector assembly comprises:
a first radiation detector for detecting the filtered radiation and generating a radiation signal in accordance with the detected radiation; and
a second radiation detector for detecting the filtered radiation and generating a radiation signal in accordance with the detected radiation,
wherein the detection of the filtered radiation of the first and second filters occurs concurrently.
